(19) United States
(12) Patent Application Publication
(10) Pub. No.: US 2025/0107704 A1
(43) Pub. Date: Apr. 3, 2025

(54) ENDOSCOPE

(71) Applicant: FUJIFILM Corporation, Tokyo (JP)

(72) Inventor: Takashi HARADA, Kanagawa (JP)

(73) Assignee: FUJIFILM Corporation, Tokyo (JP)

(21) Appl. No.: 18/900,611

(22) Filed: Sep. 27, 2024

(30) Foreign Application Priority Data
Sep. 29, 2023 (JP) 2023-169946

(51) Int. Cl. A61B 1/12; A61B 1/00

(52) U.S. Cl. CPC A61B 1/122; A61B 1/00114; A61B 1/125

(57) ABSTRACT

Provided is an endoscope in which cleaning or disinfection of an elevator housing portion can be reliably performed with a simple configuration.

An endoscope includes a distal end part formed with an elevator housing portion, an elevator disposed in the elevator housing portion, and a treatment tool insertion path communicating with a treatment tool outlet that is open to the elevator housing portion, in which the treatment tool insertion path has a diameter-enlarged portion that enlarges in diameter toward the treatment tool outlet.

### CROSS-REFERENCE TO RELATED APPLICATION

The present application claims priority under 35 U.S.C § 119 (a) to Japanese Patent Application No. 2023-169946 filed on Sep. 29, 2023, which is hereby expressly incorporated by reference, in its entirety, into the present application.

### BACKGROUND OF THE INVENTION

### 1. Field of the Invention

The present invention relates to an endoscope, and particularly relates to an endoscope comprising an elevator.

### 2. Description of the Related Art

In an endoscope, various treatment tools are inserted into a treatment tool insertion port provided in an operation part, and the treatment tool is led out from a treatment tool outlet that is open at a distal end part. Then, a treatment such as sampling or remedy is performed on a tissue inside a subject using the treatment tool led out from the treatment tool outlet. For example, a treatment tool such as a guide wire or a contrast tube is used for a duodenoscope, and a treatment tool such as a puncture needle is used for an ultrasonic endoscope.

The above-described treatment tool is displaced or bent in order to perform the treatment by facing a desired position in the subject. Therefore, a treatment tool elevation mechanism (elevator) for changing a lead-out direction of the treatment tool is provided at the distal end part of the endoscope.

In the endoscope comprising the elevator as described above, a body fluid (for example, a tissue fluid, blood, or a digestive fluid) of the subject may enter an elevator housing portion in which the elevator is housed. Therefore, the endoscope needs to be cleaned and disinfected every time it is used. Here, for the cleaning of the endoscope, for example, functional water such as strong acidic electrolyzed water or ozone water, or a cleaning liquid such as water is used. In addition, for the disinfection of the endoscope, for example, a disinfecting liquid such as a high-level disinfectant (glutaraldehyde, orthophthalaldehyde, or ethane peroxoic acid) is used. In the following description, the cleaning liquid or the disinfecting liquid is described as a liquid for cleaning or disinfection.

In a case of cleaning and disinfecting the elevator housing portion, a flow of the liquid for cleaning or disinfection may be hindered by an instrument such as the elevator disposed inside the elevator housing portion, and an area where cleaning and disinfection are insufficient may occur. In the endoscope disclosed in WO2018/145433A, in order to prevent the occurrence of the area where cleaning and disinfection are insufficient, a liquid inlet port (hereinafter, a liquid injection port) and a liquid outlet port (hereinafter, a liquid discharge port) are formed on a wall surface of the elevator housing portion. Then, a liquid is injected into the elevator housing portion from the liquid injection port and discharged from the liquid discharge port. More specifically, in WO2018/145433A, the liquid injection port is formed toward the elevator disposed in the elevator housing portion, and the liquid discharge port is formed on a wall surface on a lower side of the elevator, whereby the liquid flows to the lower side of the elevator to prevent insufficient cleaning and disinfection of the lower side of the elevator.

### SUMMARY OF THE INVENTION

In a case in which the liquid injection port is provided on the wall surface of the elevator housing portion as in WO2018/145433A, the body fluid may also enter a liquid injection path via the liquid injection port that is open to the elevator housing portion. Therefore, it is necessary to clean and disinfect the liquid injection port and the liquid injection path.

However, as described above, since the liquid injection port is formed toward the elevator, the elevator obstructs the cleaning and disinfection of the liquid injection port and the liquid injection path. For example, in a case in which cleaning is performed by inserting a cleaning brush into the liquid injection path and then pulling out the cleaning brush to a side opposite to an insertion side, the elevator obstructs the operation of the cleaning brush.

Further, the endoscope disclosed in WO2018/145433A requires a space for providing the liquid injection path. Therefore, a size (diameter) of the distal end part of the endoscope to be inserted into a body cavity of the subject is increased.

The present invention has been made in view of such circumstances, and an object of the present invention is to provide an endoscope in which an elevator housing portion can be reliably cleaned or disinfected with a simple configuration.

According to a first aspect of the present invention, there is provided an endoscope comprising: a distal end part formed with an elevator housing portion; an elevator disposed in the elevator housing portion; and a treatment tool insertion path communicating with a treatment tool outlet that is open to the elevator housing portion, in which the treatment tool insertion path has a diameter-enlarged portion that enlarges in diameter toward the treatment tool outlet.

According to a second aspect of the present invention, the endoscope according to the first aspect further comprises: a wire insertion path into which an operation wire is inserted, in which the wire insertion path and the elevator are disposed on sides opposite to each other across a partition wall, the partition wall is provided with a through-hole penetrating a space between a side where the wire insertion path is disposed and a side where the elevator is disposed, and a power transmitting member that is inserted into the through-hole and that has one end connected to the elevator and the other end connected to the operation wire is provided.

According to a third aspect of the present invention, in the endoscope according to the second aspect, the power transmitting member includes a rotation shaft to which the elevator is connected on one end side, and an elevating lever that is provided on the other end side of the rotation shaft and to which the operation wire is connected.

According to a fourth aspect of the present invention, the endoscope according to the first aspect further comprises: a wire insertion path into which an operation wire is inserted, in which the elevator is rotatably disposed in the elevator housing portion, and the operation wire is connected to the elevator.

According to a fifth aspect of the present invention, in the endoscope according to any one of the first to fourth aspects, the elevator housing portion is provided with an opening portion that has a component in a direction orthogonal to an opening direction of the treatment tool outlet and that is open in a first direction, and in a cross section of the diameter-enlarged portion along a plane determined by the opening direction of the treatment tool outlet and the first direction, an extension line of a bottom portion of the diameter-enlarged portion on a second direction side opposite to the first direction is located on the second direction side with respect to a rotation shaft of the elevator.

According to a sixth aspect of the present invention, in the endoscope according to any one of the first to fifth aspects, the elevator housing portion is provided with an opening portion that has a component in a direction orthogonal to an opening direction of the treatment tool outlet and that is open in a first direction, the elevator has a treatment tool guide surface that guides a treatment tool led out from the treatment tool outlet toward the opening portion, and in a cross section of the treatment tool insertion path along a plane determined by the opening direction of the treatment tool outlet and the first direction, an extension line of a bottom portion of a connecting portion, which is connected to a base end side of the diameter-enlarged portion, in the treatment tool insertion path on a second direction side opposite to the first direction intersects the treatment tool guide surface.

According to a seventh aspect of the present invention, in the endoscope according to any one of the first to sixth aspects, the treatment tool outlet also serves as a liquid supply port for supplying a liquid for cleaning or disinfection to the elevator.

According to an eighth aspect of the present invention, in the endoscope according to the seventh aspect, the elevator has a liquid guide surface that guides the liquid supplied to the elevator in a direction in which a rotation shaft of the elevator is provided.

According to a ninth aspect of the present invention, in the endoscope according to the eighth aspect, the liquid guide surface is provided at a position facing the treatment tool outlet in a case in which the elevator is in an elevated state.

According to the present invention, since the diameter-enlarged portion that enlarges in diameter from the treatment tool insertion path toward the treatment tool outlet is provided, the elevator housing portion can be reliably cleaned or disinfected.

### BRIEF DESCRIPTION OF THE DRAWINGS

FIG. 1 is an exploded perspective view showing a distal end part of an endoscope according to a first embodiment of the present invention.

FIG. 2 is a perspective cross-sectional view showing the distal end part of the endoscope according to the first embodiment of the present invention.

FIG. 3 is a cross-sectional view showing comparison between the distal end part of the endoscope according to the first embodiment of the present invention and a comparative example.

FIG. 4 is a diagram showing a modification example of a diameter-enlarged portion.

FIG. 5 is a cross-sectional view showing a distal end part of an endoscope according to a second embodiment of the present invention.

### DESCRIPTION OF THE PREFERRED EMBODIMENTS

Hereinafter, embodiments of an endoscope according to the present invention will be described with reference to the accompanying drawings.

### First Embodiment

An endoscope according to the present embodiment includes an operation part that is gripped by an operator and that is used for various operations, and an insertion part that is inserted into a body cavity of a subject (patient). The operation part and the insertion part are not shown.

The insertion part has an elongated shape that extends from a distal end of the operation part, and includes a soft part having flexibility, a bendable part that is bent by operating an angle knob of the operation part, and a distal end part, in this order from an operation part side (base end side).

The operation part is provided with a treatment tool inlet into which a treatment tool is inserted, and the distal end part of the insertion part is provided with a treatment tool outlet through which the treatment tool is led out. The treatment tool inlet and the treatment tool outlet are connected via a treatment tool insertion path.

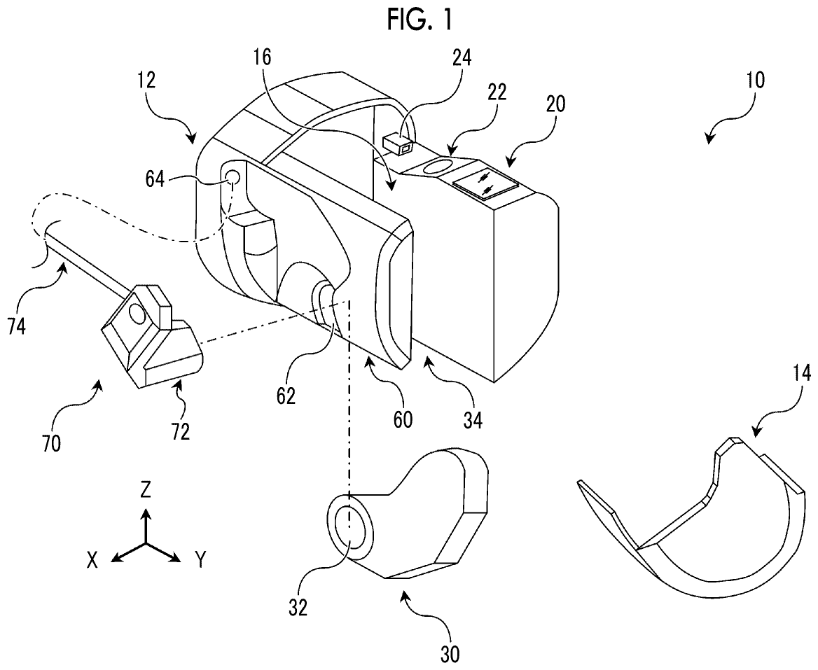

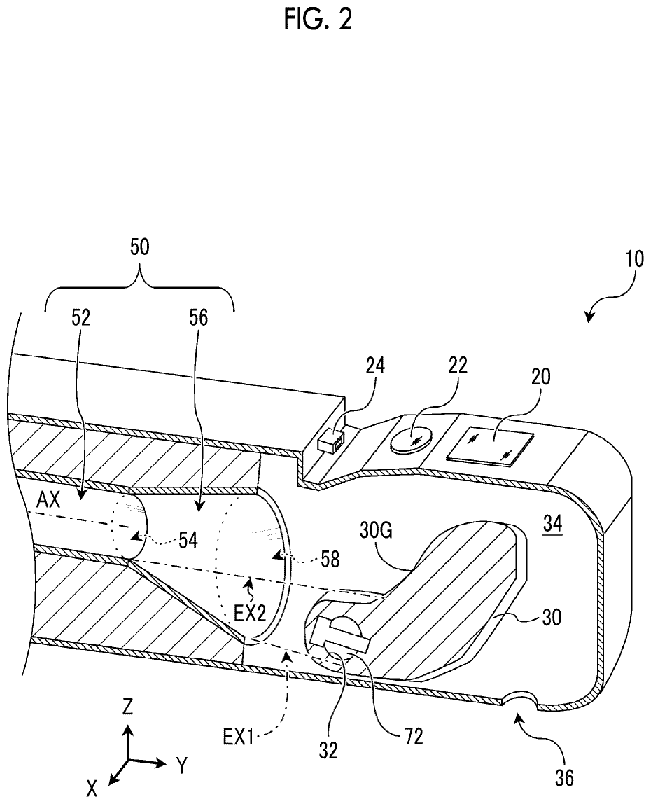

Hereinafter, a distal end part of the endoscope will be described. FIGS. 1 and 2 are views (exploded perspective view and perspective cross-sectional view, respectively) showing a distal end part of an endoscope according to a first embodiment of the present invention.

As shown in FIG. 1, a distal end part 10 includes a housing portion 12 that forms an outer wall and an internal partition wall of the distal end part 10, and a distal end cover 14 that is attached to a distal end of the housing portion 12 and that forms a part of the outer wall of the distal end part 10. The housing portion 12 and the distal end cover 14 are formed of, for example, a metal material such as stainless steel.

The housing portion 12 is formed with an illumination window 20 for illuminating an inside of the subject with illumination light guided to the housing portion 12 from a light source (not shown), an observation window 22 for imaging the inside of the subject using an imaging element housed in the housing portion 12, and an air and water supply nozzle 24 for delivering a gas or a liquid to the observation window 22.

As shown in FIG. 2, an elevator housing portion 34 in which an elevator 30 is housed is formed in the housing portion 12.

The elevator 30 is formed of, for example, a metal material such as stainless steel, and is rotatably attached about an axial direction of a rotation shaft 72, which will be described below. In the following description, a direction along the axial direction of the rotation shaft 72 is defined as an X direction, a direction in which the elevator 30 is elevated and reclined is defined as a Z direction (up-down direction), and a direction orthogonal to the ZX direction is defined as a Y direction. Here, a +Z direction and a −Z direction correspond to a first direction and a second direction, respectively.

As shown in FIG. 1, an opening portion 16 is formed in the housing portion 12. The opening portion 16 is formed in the +Z direction (first direction, upward), and has a component in a direction (Z direction) orthogonal to an opening direction of a treatment tool outlet 58. The treatment tool led out from a treatment tool insertion path 50 to the treatment tool outlet 58 is guided in the direction of the opening portion 16 by the elevator 30 in the elevator housing portion 34 and is led out from the opening portion 16 into the subject.

In addition, the housing portion 12 is provided with a partition wall 60 that constitutes a wall surface on a +X side of the elevator housing portion 34, and a through-hole 62 that penetrates a side on which a wire insertion path 64 is disposed and a side on which the elevator 30 is disposed is formed in the partition wall 60.

The rotation shaft 72 is formed through a bent portion at one end of an elevating lever 70, and an operation wire 74 is connected to the other end. The rotation shaft 72 is inserted into the through-hole 62 formed in the partition wall 60, and is inserted into a rotation shaft insertion hole 32 of the elevator 30 so as not to rotate relative to the elevator 30. The elevating lever 70 and the rotation shaft 72 function as a power transmitting member. Hereinafter, the rotation shaft 72 in a case of being inserted into the rotation shaft insertion hole 32 of the elevator 30 is also referred to as the rotation shaft 72 of the elevator 30.

The operation wire 74 is inserted into the wire insertion path 64 formed on a-Y side (base end side) of the housing portion 12, and is connected to an elevating operation lever (not shown) of the operation part of the endoscope. As shown in FIG. 1, the wire insertion path 64 and the elevator 30 are disposed on sides opposite to each other across the partition wall 60.

The treatment tool outlet 58 is formed in the elevator housing portion 34. As shown in FIG. 2, the treatment tool outlet 58 is open on a +Y side (distal end side) of the elevator housing portion 34. As described above, the treatment tool outlet 58 communicates with the treatment tool inlet of the operation part via the treatment tool insertion path 50 inserted into the insertion part of the endoscope. The treatment tool outlet 58 also serves as a liquid supply port for supplying a liquid for cleaning or disinfection to the elevator 30.

In a case of observing or treating an inside of the subject, the distal end part 10 is inserted into the subject, and the treatment tool inserted from the treatment tool inlet is led out from the treatment tool outlet 58 to the elevator housing portion 34. By operating the elevating operation lever of the operation part, the operator can displace the elevator 30 between an elevated state (elevated position) and a reclined state (reclined position) and can make the elevator 30 change a lead-out direction (lead-out angle) of the treatment tool.

On the other hand, in a case of cleaning or disinfecting the endoscope, the liquid for cleaning or disinfection is injected from the treatment tool inlet of the operation part. The liquid for cleaning or disinfection is supplied from the treatment tool outlet 58 to the elevator housing portion 34 through the treatment tool insertion path 50. The elevator housing portion 34 is provided with a liquid discharge port 36 that is open on a-Z side (second direction, downward). The liquid discharge port 36 is formed to penetrate either or both of the housing portion 12 of the distal end part 10 and the distal end cover 14, depending on a position where the liquid discharge port 36 is formed. The liquid for cleaning or disinfection is discharged to an outside of the elevator housing portion 34 via the liquid discharge port 36.

In the example shown in FIG. 2, the liquid discharge port 36 is provided at one location facing downward on an opposite side (+Y side, distal end side) of the elevator housing portion 34 with respect to the treatment tool outlet 58 which also serves as the liquid supply port, but the disposition, number, and orientation of the liquid discharge port 36 are not limited to this example. A plurality of the liquid discharge ports 36 may be provided, or the liquid discharge port 36 may be provided diagonally with respect to the treatment tool outlet 58 depending on a shape of the elevator housing portion 34.

As shown in FIG. 2, a diameter-enlarged portion 56 that expands (enlarges in diameter) toward the treatment tool outlet 58 is formed on the +Y side (distal end side) of the treatment tool insertion path 50. The diameter-enlarged portion 56 has the largest diameter at the treatment tool outlet 58. The diameter-enlarged portion 56 is not formed by chamfering the treatment tool insertion path 50, that is, by rounding a corner portion between the treatment tool insertion path 50 and the elevator housing portion 34, but is formed as described below, and is preferably formed to satisfy conditions A and B.

A portion where a portion (insertion part 52) other than the diameter-enlarged portion 56 on the-Y side (operation part side, base end side) of the treatment tool insertion path 50 and the diameter-enlarged portion 56 are connected is defined as a connecting portion 54. The diameter-enlarged portion 56 has the same diameter as the insertion part 52 at the connecting portion 54. In the example shown in FIG. 2, the diameter-enlarged portion 56 has the smallest diameter at the connecting portion 54. In the example shown in FIG. 2, the diameter-enlarged portion 56 has a substantially conical shape (oblique conical shape), but the shape of the diameter-enlarged portion 56 is not limited to the example shown in FIG. 2.

By providing such a diameter-enlarged portion 56, the treatment tool outlet 58 which also serves as the liquid supply port expands more than the treatment tool insertion path 50, so that the liquid for cleaning or disinfection can be supplied to a wide range of the elevator housing portion 34.

Further, in the present embodiment, it is preferable to configure the distal end part 10 to satisfy the following condition A or B.

### Condition A

In a cross section of the treatment tool insertion path 50 along a plane (YZ plane) determined by the Y direction (opening direction of the treatment tool outlet 58) and the Z direction (up-down direction), an extension line of a bottom portion of the diameter-enlarged portion 56 on a −Z direction (second direction) side is denoted by EX1. As shown in FIG. 2, the diameter-enlarged portion 56 is formed such that the extension line EX1 of the bottom portion is located on the-Z direction (second direction) side with respect to the rotation shaft 72 (the rotation shaft insertion hole 32) of the elevator 30. As a result, the liquid for cleaning or disinfection can be supplied to, for example, a connection point (including a gap between the rotation shaft 72 and the rotation shaft insertion hole 32) between the rotation shaft 72 and the rotation shaft insertion hole 32 in a case in which the rotation shaft 72 is inserted into the rotation shaft insertion hole 32 of the elevator 30, a sealing member (for example, an O-ring) provided between the through-hole 62 of the partition wall 60 and the rotation shaft 72, or a lower surface of the elevator 30.

### Condition B

In the cross section of the treatment tool insertion path 50 along the YZ plane, an extension line of a bottom portion of the connecting portion 54 on the-Z direction (second direction) side is denoted by EX2. As shown in FIG. 2, positions of the insertion part 52 and the elevator 30 are adjusted such that the extension line EX2 intersects a treatment tool guide surface 30G of the elevator 30. That is, the treatment tool guide surface 30G is located on the extension line EX2 or is located slightly on the-Z direction (second direction) side with respect to the extension line EX2. As a result, the treatment tool can be smoothly guided even in a case in which the diameter-enlarged portion 56 is provided.

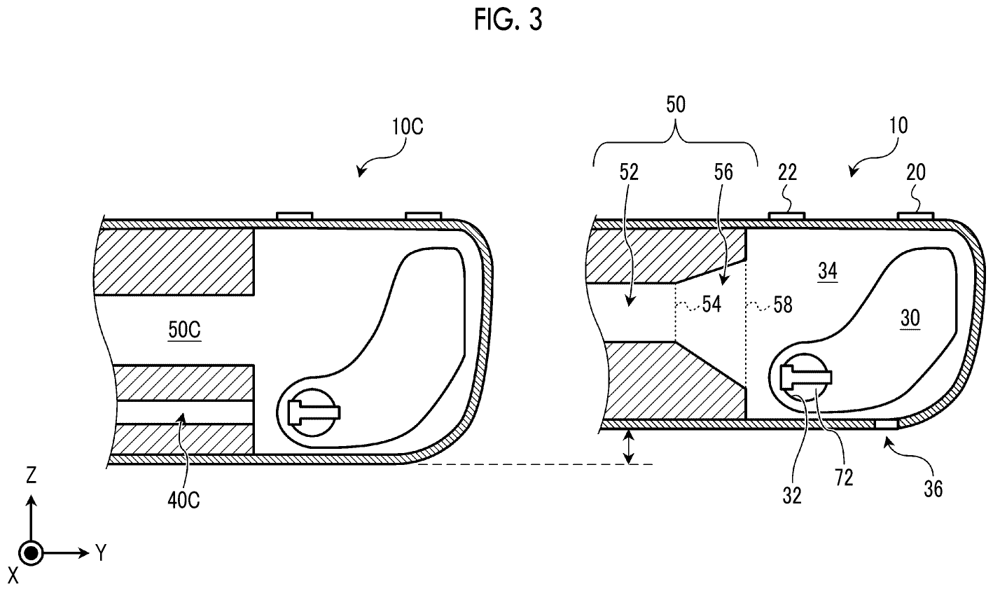

FIG. 3 is a cross-sectional view showing comparison between the endoscope according to the first embodiment of the present invention and an endoscope according to a comparative example.

As shown in FIG. 3, in a distal end part 10C of the endoscope according to the comparative example, a liquid supply path 40C for supplying the liquid for cleaning or disinfection is provided in addition to a treatment tool insertion path 50C, in order to supply a liquid to the lower side of the elevator. Since the liquid supply path 40C is provided, a size of the distal end part 10C according to the comparative example in the Z direction is increased.

On the other hand, in the distal end part 10 of the endoscope according to the present embodiment, by providing the diameter-enlarged portion 56, the liquid for cleaning or disinfection can be supplied to the lower side of the elevator 30 without providing the dedicated liquid supply path 40C. Therefore, according to the present embodiment, it is possible to reliably clean or disinfect the elevator housing portion 34 with a simpler configuration without increasing the diameter of the distal end part 10 of the endoscope.

### Modification Examples of Diameter-Enlarged Portion

The diameter-enlarged portion 56 according to the present embodiment has a substantially conical shape (truncated cone), but the present invention is not limited to this. In addition, the diameter-enlarged portion 56 may be non-axisymmetric, and an axis (central axis) of the diameter-enlarged portion 56 may be offset with respect to an axis AX of the insertion part 52 (for example, may be shifted up or down).

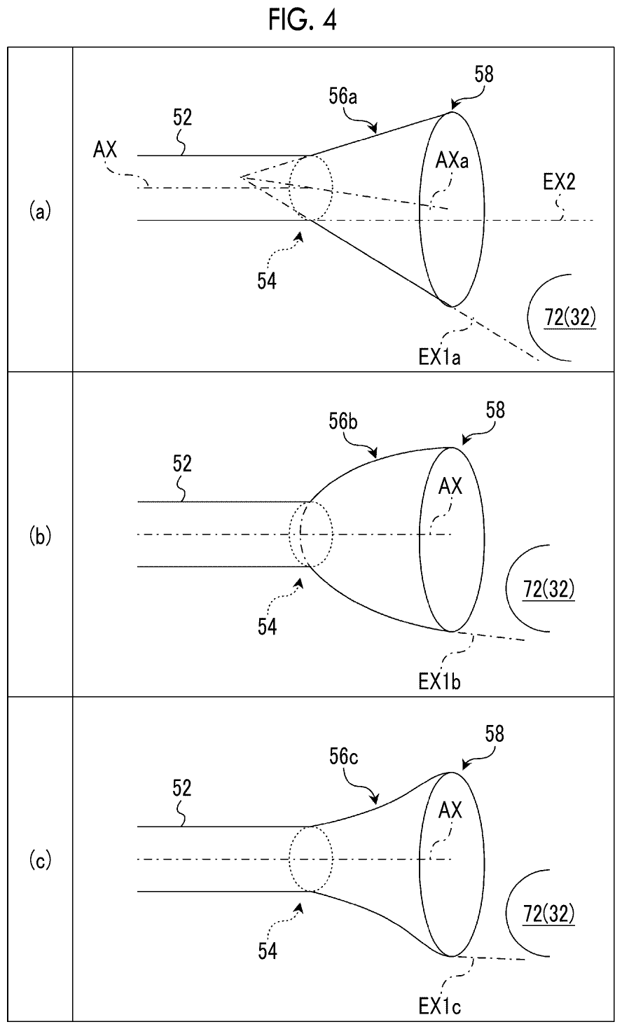

FIG. 4 is a view showing a modification example of the diameter-enlarged portion.

There is an example in which a diameter-enlarged portion 56a according to a modification example (a) has a non-axisymmetric shape. A specific shape of the diameter-enlarged portion 56a according to the modification example (a) is an oblique conical surface shape, but may be any other non-axisymmetric shape.

In the modification example (a), for example, inclination of an axis AXa of the diameter-enlarged portion 56a having the oblique conical surface shape may be adjusted depending on a positional relationship between the insertion part 52 of the treatment tool insertion path 50 and the rotation shaft 72 (the rotation shaft insertion hole 32) of the elevator 30 (for example, may be inclined to the rotation shaft 72 side of the elevator 30). As a result, as in the embodiment described above, an extension line EX1a of a longer main bus bar of the diameter-enlarged portion 56a can be located on the-Z direction (second direction) side with respect to the rotation shaft 72 of the elevator 30 (condition A).

For example, there may be a case in which it is difficult to satisfy the conditions A and B in the axisymmetric diameter-enlarged portion 56 in terms of design due to constraints of the arrangement of the components of the distal end part 10, the shape of the elevator 30, or the like. Even in such a case, by adopting the diameter-enlarged portion 56a having the non-axisymmetric shape, it is possible to satisfy the conditions A and B, and a degree of freedom in the design of the distal end part 10 is improved.

Diameter-enlarged portions 56b and 56c according to modification examples (b) and (c) have a curved surface shape in the YZ direction. Here, as the shape of the diameter-enlarged portions 56b and 56c, for example, a surface of revolution, a paraboloid, or a hyperboloid is considered.

In the modification examples (b) and (c), the cross-sectional shape is curved, but in this case, extension lines of bottom portions of the diameter-enlarged portions 56b and 56c can be tangents EX1b and EX1c in bottom portion cross sections of the diameter-enlarged portions 56b and 56c.

In the modification examples (b) and (c), central axes of the diameter-enlarged portions 56b and 56c may be offset or inclined with respect to a central axis AX of the insertion part 52 depending on the positional relationship between the insertion part 52 of the treatment tool insertion path 50 and the rotation shaft 72 (the rotation shaft insertion hole 32) of the elevator 30, or the like. In addition, the diameter-enlarged portions 56b and 56c may have a non-axisymmetric shape.

As shown in the modification examples (a) to (c), the diameter-enlarged portions (56a, 56b, and 56c) can be selected depending on the positional relationship between the insertion part 52 of the treatment tool insertion path 50 and the rotation shaft 72 (rotation shaft insertion hole 32) of the elevator 30, or the like, so that the degree of freedom in the design of the distal end part 10 is improved.

### Second Embodiment

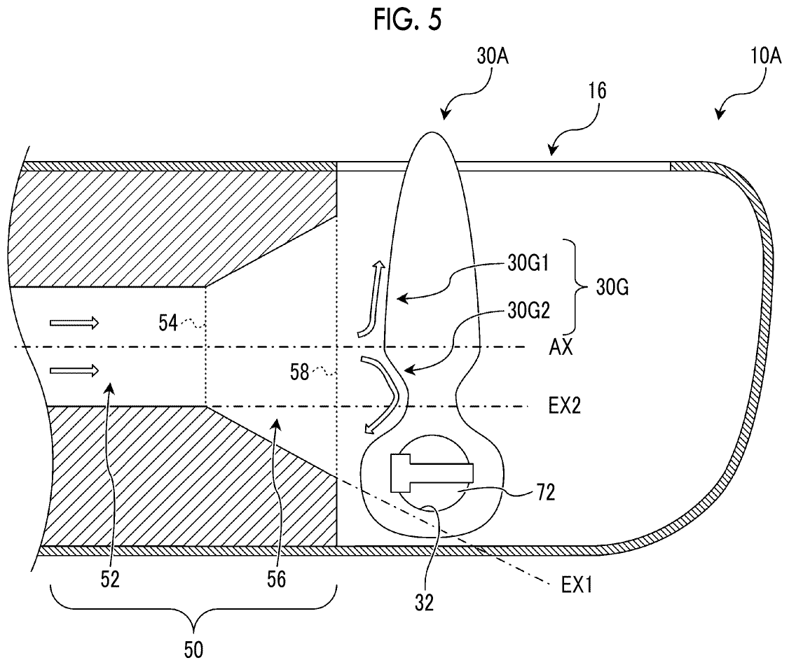

FIG. 5 is a cross-sectional view showing a distal end part of an endoscope according to a second embodiment of the present invention. FIG. 5 shows a state in which an elevator 30A is in an elevated position.

The elevator 30A according to the present embodiment includes a first surface 30G1 and a second surface 30G2 facing in different directions. The first surface 30G1 and the second surface 30G2 are also referred to as liquid guide surfaces. By providing the first surface 30G1 and the second surface 30G2, the liquid for cleaning or disinfection supplied from the treatment tool outlet 58 can flow to both the opening portion 16 side and the rotation shaft 72 (rotation shaft insertion hole 32) side.

As shown in FIG. 5, in a case in which the elevator 30 is in the elevated position, the first surface 30G1 and the second surface 30G2 are provided on an inner (−Z) side of a facing region facing the treatment tool outlet 58. The first surface 30G1 is formed obliquely upward with respect to the axis AX of the insertion part 52 of the treatment tool insertion path 50, and the second surface 30G2 is formed obliquely downward with respect to the axis AX of the insertion part 52 of the treatment tool insertion path 50. That is, since the first surface 30G1 is inclined to the opening portion 16 side of the elevator housing portion 34, a liquid that has hit the first surface 30G1 can be guided to the opening portion 16 side of the elevator housing portion 34. In addition, since the second surface 30G2 is inclined to the rotation shaft 72 side of the elevator 30A, a liquid that has hit the second surface 30G2 can be guided to the rotation shaft 72 side of the elevator 30A.

According to the present embodiment, by providing the liquid guide surfaces (first surface 30G1 and second surface 30G2) on the elevator 30A, the elevator housing portion 34 can be more reliably cleaned or disinfected.

A shape of the liquid guide surface is not limited to the example shown in FIG. 5. The liquid guide surface may include, for example, a plurality of surfaces of which a direction in which the liquid is guided is adjusted according to the arrangement of the components of the distal end part 10. For example, a plurality of liquid guide surfaces capable of guiding the liquid to a region on an opposite side of the treatment tool outlet 58 with respect to the components obstructing the liquid flow may be provided.

In addition, in each of the above-described embodiments, the diameter-enlarged portion 56 enlarges in diameter in both the +Z direction and the-Z direction (both the up and down directions), but may enlarge in diameter only on the-Z direction (second direction) side.

In addition, in each of the above-described embodiments, the elevator 30 is operated via the elevating lever 70, but the present invention is not limited to this. For example, the above-described embodiments can be applied to an endoscope of a type in which the operation wire 74 is directly connected to the elevator 30 without providing the partition wall 60 or to other types of endoscopes (for example, JP1999-299728A (JP-H11-299728A), JP2012-040258A, and WO2021/033641A).

### EXPLANATION OF REFERENCES

10: distal end part
12: housing portion
14: distal end cover
16: opening portion
20: illumination window
22: observation window
24: air and water supply nozzle
30: elevator
30A: elevator
30G: treatment tool guide surface
30G1: first surface
30G2: second surface
32: rotation shaft insertion hole
34: elevator housing portion
36: liquid discharge port
50: treatment tool insertion path
52: insertion part
54: connecting portion
56: diameter-enlarged portion
56a: diameter-enlarged portion
56b: diameter-enlarged portion
56c: diameter-enlarged portion
58: treatment tool outlet
60: partition wall
62: through-hole
64: wire insertion path
70: elevating lever
72: rotation shaft
74: operation wire
EX1: extension line
EX1a: extension line
EX1b: extension line
EX1c: extension line
EX2: extension line
AX: axis
AXa: axis
10C: distal end part
40C: liquid supply path
50C: treatment tool insertion path

What is claimed is:

1. An endoscope comprising:
a distal end part formed with an elevator housing portion;
an elevator disposed in the elevator housing portion; and
a treatment tool insertion path communicating with a treatment tool outlet that is open to the elevator housing portion,
wherein the treatment tool insertion path has a diameter-enlarged portion that enlarges in diameter toward the treatment tool outlet.

2. The endoscope according to claim 1, further comprising:
a wire insertion path into which an operation wire is inserted,
wherein the wire insertion path and the elevator are disposed on sides opposite to each other across a partition wall,
the partition wall is provided with a through-hole penetrating a space between a side where the wire insertion path is disposed and a side where the elevator is disposed, and
a power transmitting member that is inserted into the through-hole and that has one end connected to the elevator and the other end connected to the operation wire is provided.

3. The endoscope according to claim 2,
wherein the power transmitting member includes
a rotation shaft to which the elevator is connected on one end side, and
an elevating lever that is provided on the other end side of the rotation shaft and to which the operation wire is connected.

4. The endoscope according to claim 1, further comprising:
a wire insertion path into which an operation wire is inserted,
wherein the elevator is rotatably disposed in the elevator housing portion, and
the operation wire is connected to the elevator.

5. The endoscope according to any one of claim 1,
wherein the elevator housing portion is provided with an opening portion that has a component in a direction orthogonal to an opening direction of the treatment tool outlet and that is open in a first direction, and
in a cross section of the diameter-enlarged portion along a plane determined by the opening direction of the treatment tool outlet and the first direction, an extension line of a bottom portion of the diameter-enlarged portion on a second direction side opposite to the first direction is located on the second direction side with respect to a rotation shaft of the elevator.

6. The endoscope according to claim 1,
wherein the elevator housing portion is provided with an opening portion that has a component in a direction orthogonal to an opening direction of the treatment tool outlet and that is open in a first direction,
the elevator has a treatment tool guide surface that guides a treatment tool led out from the treatment tool outlet toward the opening portion, and
in a cross section of the treatment tool insertion path along a plane determined by the opening direction of the treatment tool outlet and the first direction, an extension line of a bottom portion of a connecting portion, which is connected to a base end side of the diameter-enlarged portion, in the treatment tool insertion path on a second direction side opposite to the first direction intersects the treatment tool guide surface.

7. The endoscope according to claim 1,
wherein the treatment tool outlet also serves as a liquid supply port for supplying a liquid for cleaning or disinfection to the elevator.

8. The endoscope according to claim 7,
wherein the elevator has a liquid guide surface that guides the liquid supplied to the elevator in a direction in which a rotation shaft of the elevator is provided.

9. The endoscope according to claim 8,
wherein the liquid guide surface is provided at a position facing the treatment tool outlet in a case in which the elevator is in an elevated state.
